(19) United States
(12) Patent Application Publication
(10) Pub. No.: US 2026/0318726 A1
(43) Pub. Date: Oct. 15, 2026

(54) METHODS OF PREDICTING CONDITIONS INDICATIVE OF HYDRATE FORMATION WITHIN A FLUID CONDUIT, METHODS OF MONITORING FLUID FLOW WITHIN A FLUID CONDUIT FOR HYDRATE FORMATION, AND HYDROCARBON CONVEYANCE STRUCTURES THAT PERFORM THE METHODS

(71) Applicant: ExxonMobil Technology and Engineering Company, Spring, TX (US)

(72) Inventor: Douglas J. TURNER, Kingwood, TX (US)

(21) Appl. No.: 19/145,620

(22) PCT Filed: Dec. 20, 2023
(86) PCT No.: PCT/US2023/085204

§ 371 Date: Jul. 3, 2025

Related U.S. Application Data
(60) Provisional application No. 63/479,023, filed on Jan. 9, 2023.

(51) Int. Cl. G01F 1/68; E21B 47/103; G01N 30/02; G01N 30/88; G01N 33/28

(52) U.S. Cl. CPC G01F 1/68; E21B 47/103; G01N 30/88; G01N 33/2823; E21B 2200/20; G01N 2030/025; G01N 2030/8854

(57) ABSTRACT

Methods of predicting conditions indicative of hydrate formation within a fluid conduit, methods of monitoring fluid flow within a fluid conduit for hydrate formation, and hydrocarbon conveyance systems that perform the methods are disclosed herein. The methods of predicting conditions include determining a plurality of corresponding flow conditions for each temperature of a plurality of different temperatures and applying a plurality of test conditions to a hydrate formation model. These methods also include generating, from the hydrate formation model, a hydrate formation model output and training a machine learning model utilizing the hydrate formation model output. The methods of monitoring fluid flow include detecting a temperature and a composition of the fluid stream and providing the temperature and the composition of the fluid stream to a machine learning model. These methods also include predicting a statistical probability of hydrate formation, within the fluid conduit, via the machine learning model.

### CROSS-REFERENCE TO RELATED APPLICATION

This application claims the benefit of U.S. Provisional Application Ser. No. 63/479,023, entitled “METHODS OF PREDICTING CONDITIONS INDICATIVE OF HYDRATE FORMATION WITHIN A FLUID CONDUIT, METHODS OF MONITORING FLUID FLOW WITHIN A FLUID CONDUIT FOR HYDRATE FORMATION, AND HYDROCARBON CONVEYANCE STRUCTURES THAT PERFORM THE METHODS,” filed Jan. 9, 2023, the disclosure of which is hereby incorporated by reference in its entirety.

### FIELD OF THE DISCLOSURE

The present disclosure relates generally to methods of predicting conditions indicative of hydrate formation within a fluid conduit, to methods of monitoring fluid flow within a fluid conduit, and to hydrocarbon conveyance structures that perform the methods.

### BACKGROUND OF THE DISCLOSURE

Hydrates may form within fluid streams that contain water and hydrocarbons. Hydrate formation is difficult to monitor, and conventional methodologies for detecting hydrate formation rely upon detection of pressure drops associated with flow occlusion that accompanies hydrate formation. Such detection methodologies often are unable to detect the hydrate formation early enough to prevent complete, or near complete, blockage of a corresponding fluid conduit.

Hydrate formation historically has been mitigated via injection of an inhibitor into the fluid stream. While inhibitor injection is effective at decreasing a potential for hydrate formation, it often is ineffective at removing already formed hydrate from the fluid conduit. This, coupled with the inaccuracy and time lag of conventional hydrate blockage formation predictions, dictates that it often is necessary to inject a significant excess of the inhibitor in order to provide a desired operating margin for hydrate formation. Injection of excess inhibitor increases the overall costs associated with conveying the fluid stream from one location to another.

It would be beneficial to more accurately monitor hydrate formation, to more quickly detect hydrate formation, and to decrease inhibitor usage. Thus, there exists a need for improved methods of predicting conditions indicative of hydrate formation within a fluid conduit, for improved methods of monitoring fluid flow within a fluid conduit, and/or for improved structures that perform the methods.

### SUMMARY OF THE DISCLOSURE

Methods of predicting conditions indicative of hydrate formation within a fluid conduit, methods of monitoring fluid flow within a fluid conduit for hydrate formation, and hydrocarbon conveyance systems that perform the methods are disclosed herein. The methods of predicting conditions include determining a plurality of corresponding flow conditions for each temperature of a plurality of different temperatures. These methods also include applying a plurality of test conditions to a hydrate formation model. Each test condition includes a corresponding temperature of the plurality of different temperatures and the plurality of corresponding flow conditions of the fluid stream for the corresponding temperature. These methods also include generating, from the hydrate formation model, a hydrate formation model output that predicts liquid phase composition, gaseous phase composition, and hydrate phase composition within the fluid stream for each test condition. These methods also include training a machine learning model, and utilizing the hydrate formation model output, such that, for a given temperature of the fluid stream, a given stream pressure of the fluid stream, and a given composition of the fluid stream, the machine learning model selectively predicts combinations of the liquid phase composition and the gaseous phase composition indicative of the presence of a hydrate phase within the fluid conduit.

The methods of monitoring fluid flow include detecting a temperature of the fluid stream and a composition of the fluid stream, and the methods of monitoring fluid flow further include providing the temperature of the fluid stream and the composition of the fluid stream to a machine learning model. These methods also include predicting a statistical probability of hydrate formation, within the fluid conduit, via the machine learning model.

### BRIEF DESCRIPTION OF THE DRAWINGS

FIG. 1 is a schematic illustration of examples of a hydrate conveyance structure that may perform methods, according to the present disclosure.

FIG. 2 is a plot illustrating an example of pressure drop as a function of time, within a fluid conduit, and illustrates a need for methods, according to the present disclosure.

FIG. 3 is a pressure-temperature phase diagram for hydrate formation within a fluid conduit and illustrates a need for methods, according to the present disclosure.

FIG. 4 is a bar chart illustrating an example of relative compositions of components of a fluid stream that are not indicative of hydrate formation as predicted by methods, according to the present disclosure.

FIG. 5 is a bar chart illustrating an example of relative compositions of components of a fluid stream that are indicative of hydrate formation as predicted by methods, according to the present disclosure.

FIG. 6 is a flowchart depicting examples of methods of predicting conditions indicative of hydrate formation within a fluid conduit, according to the present disclosure.

FIG. 7 is a flowchart depicting examples of methods of monitoring flow of a fluid stream within a fluid conduit for hydrate formation, according to the present disclosure.

### DETAILED DESCRIPTION AND BEST MODE OF THE DISCLOSURE

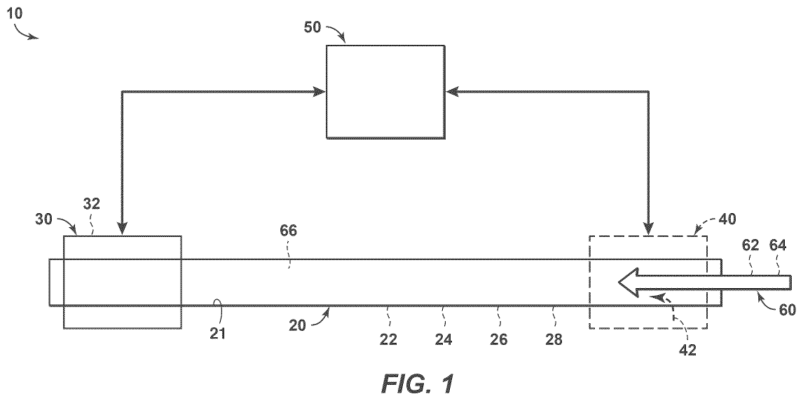

FIGS. 1-7 provide examples of hydrocarbon conveyance structures 10, of conditions that may be present within hydrocarbon conveyance structures 10, and/or of methods 100 and 200, according to the present disclosure. Elements, components, and/or features that are discussed herein with reference to one or more of FIGS. 1-7 may be included in and/or utilized with any of FIGS. 1-7 without departing from the scope of the present disclosure. In general, elements that are likely to be included in a particular embodiment are illustrated in solid lines, while elements that are optional are illustrated in dashed lines. However, elements that are shown in solid lines may not be essential to all embodiments and, in some embodiments, may be omitted without departing from the scope of the present disclosure.
FIG. 1 is a schematic illustration of examples of a hydrate conveyance structure 10 that may perform methods 100 and 200, according to the present disclosure. As illustrated in solid lines in FIG. 1, hydrocarbon conveyance structure 10 includes a fluid tubular 20 that defines a fluid conduit 21. The fluid conduit contains a fluid stream 60 that includes water 62 and hydrocarbons 64 and that also may contain other fluids 68, such as carbon dioxide, hydrogen sulfide, nitrogen, and/or argon. Examples of hydrocarbon conveyance structure 10 include a pipeline 22, a process tubular 24, a subterranean tubular 26, and/or a hydrocarbon well 28. Examples of pipeline 22 include a hydrocarbon pipeline, a subterranean hydrocarbon pipeline, a subsea hydrocarbon pipeline, and/or a surface hydrocarbon pipeline. When hydrocarbon conveyance structure 10 includes hydrocarbon well 28, the hydrocarbon well may include a wellbore that extends within a subsurface region, and fluid tubular 20 may extend within the wellbore.

As discussed, mixtures of water 62 and hydrocarbons 64 may form, or may be susceptible to forming, hydrates 66 within fluid conduit 21. Hydrates 66 may form if and/or when conditions, such as temperature and/or pressure, within the fluid conduit are conducive to and/or encourage such hydrate formation. Hydrates 66 are solids that may impede, block, and/or occlude fluid flow within fluid conduit 21. As such, it may be desirable to avoid hydrate formation within the fluid conduit.

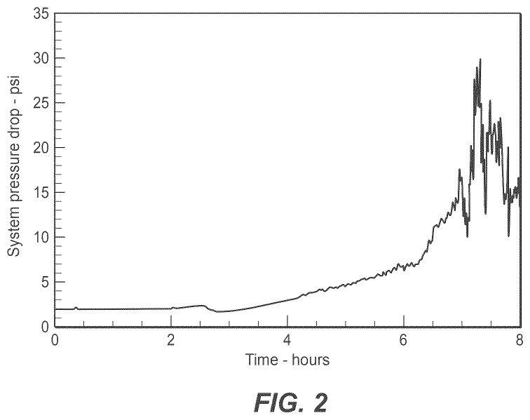

As also discussed, conventional methodologies for detecting hydrate formation generally rely upon detection of pressure drops that are associated with and/or produced by the flow occlusion that accompanies hydrate formation. While such conventional methodologies may be beneficial in indicating that hydrate formation already has occurred, they often are ineffective at detecting hydrate formation quickly enough to permit mitigation of the hydrate formation and/or elimination of the hydrates from the fluid stream. As an example, FIG. 2 is a plot illustrating an example of pressure drop as a function of time, within a fluid conduit. As may be seen from FIG. 2, the pressure drop often increases quite rapidly. This rapid increase in pressure drop may be the result of large quantities of hydrate that may form on the inner wall of fluid tubular 20 and subsequently may slough off of the wall, leading to an accumulation of hydrates at various locations within the fluid conduit and to the accompanying increase in pressure drop. Stated differently, because conventional methodologies generally rely upon detection of pressure drops within the fluid conduit, these conventional methodologies typically do not indicate that hydrate formation has occurred until after a large quantity of hydrates is present within the fluid conduit. This large quantity of hydrates often will occlude the fluid conduit in a manner that cannot readily be mitigated utilizing conventional methodologies.

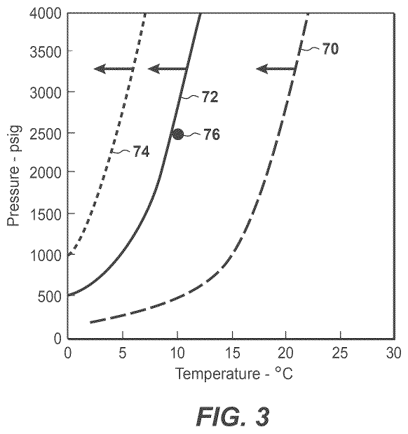

As also discussed, it is common to utilize an inhibitor to decrease the potential for hydrate formation within the fluid conduit. As an example, and as illustrated in dashed lines in FIG. 1, an inhibitor injection system 40 may be utilized to inject an inhibitor 42 into fluid conduit 21. The presence of inhibitor 42 decreases the potential for hydrate formation within the fluid conduit and/or changes conditions under which hydrate can and/or will form within the fluid conduit. This is illustrated by FIG. 3, which is a pressure-temperature phase diagram for hydrate formation. In FIG. 3, and for a given composition of the fluid stream, the curve at 70 indicates the uninhibited hydrate formation curve, with hydrate forming for combinations of pressure and temperature that are to the left of the curve, as indicated by the arrow. In addition, the point at 76 indicates the desired operating pressure and temperature for the fluid stream. Without addition of the inhibitor, hydrates will form within the fluid stream at point 76.

In order to avoid hydrate formation, the inhibitor is injected into the fluid stream. Under well-understood and well-controlled flow conditions, it would be desirable to inject inhibitor such that the hydrate formation curve is immediately to the left of operating point 76, such as may be provided by the inhibited curve that is indicated at 72. However, in practice, it generally is necessary to provide increased margin in order to avoid unexpected hydrate formation, such as by injecting excess inhibitor such that the hydrate formation curve is farther to the left of operating point 76. This is illustrated schematically in FIG. 3 by the inhibited curve that is indicated at 74. This injection of excess inhibitor represents an additional cost associated with conveyance of the fluid stream through the fluid conduit. As discussed in more detail herein, hydrocarbon conveyance structures 10 and/or methods 100 and 200, according to the present disclosure, provide a mechanism via which the margin may be decreased while still providing a desired level of protection from hydrate formation. This may permit hydrocarbon conveyance structures 10, according to the present disclosure, to operate with lower inhibitor consumption and/or at a lower cost when compared to conventional hydrocarbon conveyance structures.

As illustrated in solid lines in FIG. 1, hydrocarbon conveyance structures 10 include a detection device 30, which may be configured to detect a temperature of fluid stream 60, a plurality of different temperatures of fluid stream 60, a pressure of fluid stream 60, a plurality of different pressures of fluid stream 60, a composition of fluid stream 60, and/or a plurality of different flow conditions of fluid stream 60. Examples of detection device 30 include a temperature sensor, a pressure sensor, a composition sensor, a flow meter, a flow regime sensor, and/or a gas chromatograph.

As also illustrated in solid lines in FIG. 1, hydrocarbon conveyance structures 10 include a controller 50. Controller 50 is adapted, configured, designed, and/or programmed to control the operation of hydrocarbon conveyance structures 10 according to and/or utilizing methods 100 and/or methods 200, which are disclosed herein.

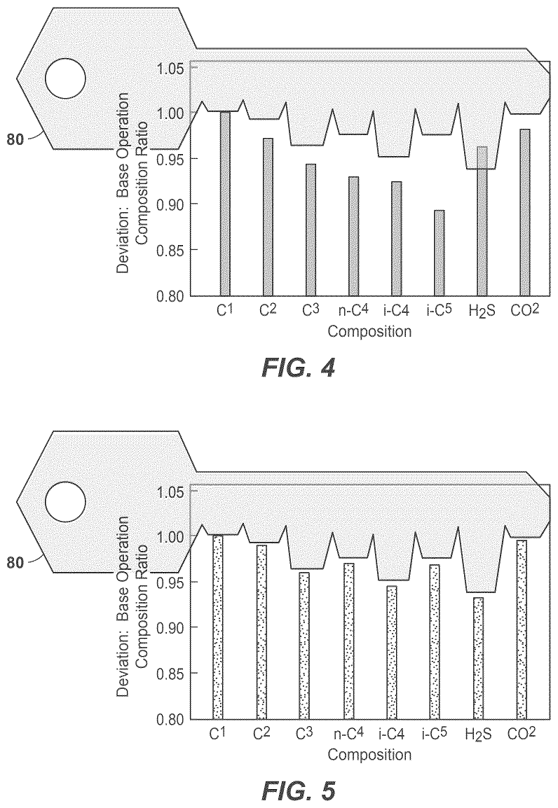

As an example, methods 100 may be utilized to produce and/or generate a flow condition key, such as may be illustrated in FIGS. 4-5 and indicated at 80. The flow condition key may indicate compositions of the fluid stream, for a given temperature and pressure of the fluid stream, that are indicative of hydrate formation within the fluid stream. In addition, methods 200 may be utilized to compare a current composition of the fluid stream, at the temperature and pressure of the fluid stream, to the flow condition key in order to determine a likelihood of hydrate formation within the fluid stream.

This is illustrated by FIGS. 4-5. More specifically, and in the example of FIG. 4, flow condition key 80 is mismatched from the illustrated compositions, thereby indicating that hydrate formation within the fluid stream is unlikely. In contrast, and in the example of FIG. 5, flow condition key 80 is matched to the illustrated compositions, thereby indicating that hydrate formation within the fluid stream is likely and/or is occurring. An amount of mismatch between the flow condition key and the composition of the fluid stream may, in some examples, indicate the probability for hydrate formation, with relatively larger mismatch magnitudes indicating a relatively lower probability of hydrate formation.

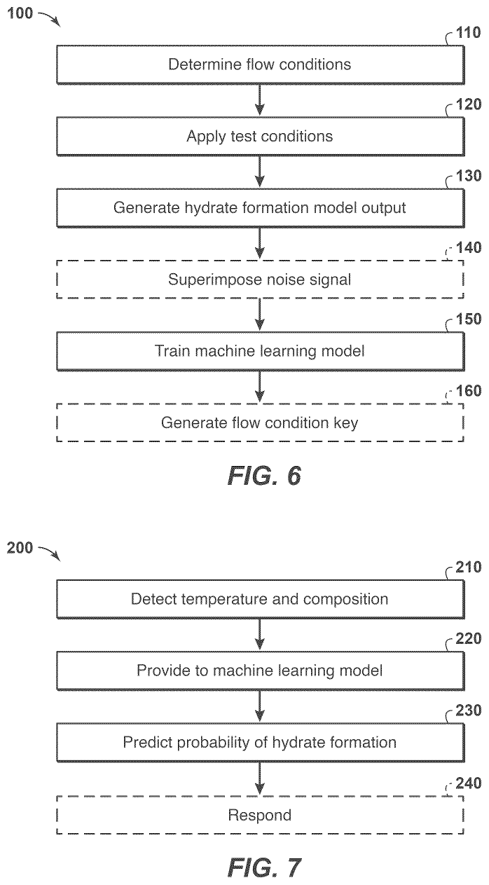

FIG. 6 is a flowchart depicting examples of methods 100 of predicting conditions indicative of hydrate formation within a fluid conduit, according to the present disclosure. The fluid conduit contains a fluid stream that includes water and hydrocarbons. Examples of the fluid conduit are disclosed herein with reference to fluid conduit 21, which may be formed and/or defined by a fluid tubular 20. Examples of the fluid stream are disclosed herein with reference to fluid stream 60.

Methods 100 include determining flow conditions at 110, applying test conditions at 120, and generating a hydrate formation model output at 130. Methods 100 may include superimposing a noise signal at 140, and methods 100 include training a machine learning model at 150. Methods 100 also may include generating a flow condition key at 160.

Determining flow conditions at 110 includes determining a plurality of corresponding flow conditions of the fluid stream for each temperature of a plurality of different temperatures of the fluid stream. Stated differently, the determining at 110 includes determining the plurality of corresponding flow conditions, which may be at least partially different at each temperature, at the plurality of different temperatures of the fluid stream.

The plurality of different temperatures may include and/or be any suitable plurality of distinct, or different, temperatures of the fluid stream, such as may be exhibited and/or experienced, by the fluid stream, during flow of the fluid stream within the fluid conduit. As an example, the plurality of different temperatures may include a plurality of selected, chosen, and/or predetermined temperatures of the fluid stream. As another example, the plurality of different temperatures may include a plurality of measured temperatures, such as may be measured within and/or of the fluid stream.

It is within the scope of the present disclosure that the fluid stream may have, exhibit, and/or experience a stream temperature variation that defines a stream temperature range. The stream temperature range may extend between a minimum stream temperature and a maximum stream temperature. The steam temperature variation may, in some examples, include and/or be a naturally occurring stream temperature variation, such as may be a result of naturally occurring variation in an ambient temperature within an environment that surrounds the fluid conduit. Examples of the stream temperature variation include day-to-night stream temperature variation, day-to-day stream temperature variation, and/or seasonal stream temperature variation.

With this in mind, the plurality of different temperatures may be based, at least in part, on the stream temperature variation and/or may be within the stream temperature variation. Stated differently, a temperature range of the plurality of different temperatures may be based, at least in part, on the stream temperature range and/or the temperature range of the plurality of different temperatures may be equal, or at least substantially equal, to the stream temperature range.

The instant application recognizes that hydrate formation, including the presence, or absence, of hydrates within the fluid conduit and/or a chemical composition of formed hydrates, when present, varies with temperature. As such, the above-described naturally occurring stream temperature variation will produce naturally occurring variations in both the presence, or absence, of hydrates within the fluid conduit and/or within the chemical composition of the formed hydrates. This will produce a naturally occurring variation within the chemical composition of other phases of the fluid stream, which also are present within the fluid conduit, thereby permitting methods 100 to predict conditions indicative of hydrate formation more quickly and/or more accurately when compared to conventional methodologies for detecting hydrate formation.

The plurality of corresponding flow conditions may include and/or be any suitable flow conditions for the fluid stream. As an example, the plurality of corresponding flow conditions may include a parameter related to motion of the fluid stream within the fluid conduit. Examples of the parameter related to motion of the fluid stream within the fluid conduit include a volumetric flow rate of the fluid stream, a flow velocity of the fluid stream, and/or a flow regime of the fluid stream. Examples of the flow regime of the fluid stream include laminar flow and/or turbulent flow. The parameter related to motion of the fluid stream may be indicative of the probability of hydrate formation withing the fluid conduit, such as via being indicative of shear and/or abrasive forces, which may be present within the fluid conduit, that may change a size of any hydrate particulate formed within the fluid conduit and/or that may change a likelihood for previously formed hydrate to dissolve and/or to disintegrate within the fluid conduit. Additionally or alternatively, the parameter related to motion of the fluid stream may be indicative of the probability that formed hydrate blocks and/or occludes the fluid conduit, such as via being indicative of a likelihood for formed hydrate to slough off of the walls of the fluid tubular that defines the fluid conduit.

As another example, the plurality of corresponding flow conditions may include a stream pressure of the fluid stream. As discussed herein with reference to FIG. 3, both the stream temperature of the fluid stream and the stream pressure of the fluid stream dictate phase behavior of the fluid stream. Thus, the stream pressure of the fluid stream may be indicative of the likelihood of hydrate formation within the fluid conduit.

As another example, the plurality of corresponding flow conditions may include a stream composition of the fluid stream. The stream composition of the fluid stream may include a hydrocarbon fraction of the fluid stream and/or an aqueous fraction of the fluid stream. The hydrocarbon fraction of the fluid stream and/or the aqueous fraction of the fluid stream may be defined and/or quantified in any suitable manner, such as via any suitable mass fraction, mole fraction, and/or volume fraction of the fluid stream. In general, the hydrocarbon fraction of the fluid stream may include and/or be an overall hydrocarbon fraction of the fluid stream, such as may be present in a combination of any solid, liquid, and/or gas phases present within the fluid stream. Similarly, the aqueous fraction of the fluid stream may include and/or be an overall aqueous fraction of the fluid stream, such as may be present in the combination of any solid, liquid, and/or gas phases present within the fluid stream.

The hydrocarbon fraction of the fluid stream may include, or further may be separated into, any suitable specific hydrocarbon composition, which may be present within the fluid stream. Examples of the hydrocarbon fraction of the fluid stream include a methane fraction of the fluid stream, an ethane fraction of the fluid stream, a propane fraction of the fluid stream, a butane fraction of the fluid stream, and/or a pentane fraction of the fluid stream. In some examples, the hydrocarbon fraction of the fluid stream may include, or may be separated into, at least 2, at least 3, at least 4, or all 5 of the methane fractions of the fluid stream, the ethane fraction of the fluid stream, the propane fraction of the fluid stream, the butane fraction of the fluid stream, and/or the pentane fraction of the fluid stream.

The stream composition of the fluid stream additionally or alternatively may include one or more other fluids. As examples, the stream composition may include a hydrogen sulfide fraction of the fluid stream, a carbon dioxide fraction of the fluid stream, a nitrogen fraction of the fluid stream, and/or an argon fraction of the fluid stream. These additional fluids, when present, may change the phase behavior of the fluid stream with respect to hydrate formation.

The stream composition of the fluid stream additionally or alternatively may include an inhibitor fraction of the fluid stream. As discussed in more detail herein, an inhibitor may be injected into the fluid stream to decrease the potential for hydrate formation of the fluid stream and/or to change the phase behavior of the fluid stream with respect to hydrate formation. With this in mind, the plurality of corresponding flow conditions additionally or alternatively may include a rate of inhibitor injection into the fluid stream. Examples of the inhibitor are disclosed herein with reference to inhibitor 42.

It is within the scope of the present disclosure that the stream composition of the fluid stream may change and/or vary with time, thereby changing and/or varying the probability of hydrate formation within the fluid conduit at and/or for a given temperature of the fluid stream and/or a given pressure of the fluid stream. As an example, the fluid stream may be conveyed through the fluid conduit from a fluid source, and a composition of fluid within the fluid source may change and/or vary with time. As another example, the fluid stream may be conveyed through the fluid conduit from a plurality of different fluid sources, each of which may include a different composition of fluid, and a proportion of the fluid stream from one or more fluid source of the plurality of different fluid sources may vary with time. As a more specific example, the fluid stream may be conveyed through the fluid conduit from a plurality of different hydrocarbon wells, and a well allocation for the plurality of different hydrocarbon wells may vary with time. With the above in mind, the ability of methods 100 to predict conditions indicative of hydrate formation based upon the fluid composition of the fluid stream and/or changes in the fluid composition of the fluid stream may provide significant benefits over conventional methodologies for detecting hydrate formation.

Applying test conditions at 120 may include applying a plurality of test conditions to a hydrate formation model. Each test condition of the plurality of test conditions may differ from each other test condition of the plurality of test conditions. Each test condition may include a corresponding temperature of the plurality of different temperatures. Each test condition also may include the plurality of corresponding flow conditions of the fluid stream for the corresponding temperature. State differently, each test condition may define a corresponding temperature and a corresponding set of flow conditions for the fluid stream. The hydrate formation model may include and/or be any suitable model, computer model, mathematical description, and/or algorithm that may be utilized to predict hydrate formation based upon the corresponding temperature and the plurality of corresponding flow conditions. An example of the hydrate formation model includes a flash-flow model of the fluid stream.

Generating the hydrate formation model output at 130 may include generating the hydrate formation model output from the hydrate formation model. The hydrate formation model output may predict a composition of various phases, which may be present within the fluid stream, for each test condition. As examples, the hydrate formation model output may predict a liquid phase composition, a gas phase composition, and/or a hydrate phase composition of the fluid stream for each test condition. As a specific example, the hydrate formation model may be configured to calculate the hydrate formation model output, including the liquid phase composition, the gas phase composition, and/or the hydrate phase composition, for each test condition. The calculation may be based, at least in part, on the corresponding temperature and the plurality of corresponding flow conditions for each test condition.

The hydrate formation model output may predict the presence of only 1 phase, of 2 phases, or of 3 phases, depending on the test condition. As an example, the hydrate formation model output may predict finite values for the liquid phase composition, the gas phase composition, and/or the hydrate phase composition when the given phase is predicted to be present within the fluid stream. As another example, the hydrate formation model may predict the absence of the liquid phase, or of the liquid phase composition, the absence of the gas phase, or of the gas phase composition, and/or the absence of the hydrate phase, or of the hydrate phase composition, when the given phase is predicted to be absent from the fluid stream.

Superimposing the noise signal at 140 may include superimposing the noise signal on the hydrate formation model output. This may include superimposing the noise signal to introduce, produce, and/or generate variation, or random variation, within the hydrate formation model output. This may increase an accuracy and/or a robustness of the machine learning model that is trained utilizing the hydrate formation model output, which includes the noise signal, and/or during the training at 150.

The superimposing at 140 may include superimposing any suitable noise signal on the hydrate formation model output. As an example, the superimposing at 140 may include superimposing a Gaussian noise signal on the hydrate formation model output. As another example, the superimposing at 140 may include superimposing a noise signal that is based expected variability, naturally occurring variability, and/or measurement variability for one or more of the plurality of corresponding flow conditions determined during the determining at 110.

Training the machine learning model at 150 may include training the machine learning model utilizing the hydrate formation model output. This may include training such that, for a given temperature of the fluid stream, a given pressure of the fluid stream, and/or a given composition of the fluid stream, the machine learning model selectively predicts combinations of the liquid phase composition and of the gas phase composition that are indicative of the presence of a hydrate phase within the fluid conduit. Subsequent to the training at 150, the machine learning model may be configured to predict a statistical probability of hydrate formation, within the fluid conduit, for a given combination of temperature and the plurality of corresponding flow conditions.

The training at 150 may be accomplished in any suitable manner. As an example, the training at 150 may include providing the hydrate formation model output to the machine learning model as training data.

Examples of the machine learning model include a supervised machine learning algorithm, a semi-supervised machine learning algorithm, an unsupervised machine learning algorithm, and/or a reinforcement machine learning algorithm.

Generating the flow condition key at 160 may include generating a flow condition key that predicts a statistical probability of hydrate formation within the fluid conduit for a given temperature of the fluid stream and a given composition of the fluid stream. Stated differently, the flow condition key may be compared to the composition of the fluid stream, at the given temperature, to provide an indication and/or an estimate of the statistical probability that hydrates are present within the fluid conduit. Examples of the flow condition key are disclosed herein with reference to flow condition key 80, and FIGS. 4-5, which are discussed in more detail herein, illustrate comparison of the flow condition key to the composition of the fluid stream.

The generating at 160 may be performed in any suitable manner and/or with any suitable timing during methods 100. As an example, the generating at 160 may include generating the flow condition key with, via, and/or utilizing the machine learning model. As another example, the generating at 160 may be performed subsequent to and/or responsive to the training at 150.

FIG. 7 is a flowchart depicting examples of methods 200 of monitoring flow of a fluid stream within a fluid conduit for hydrate formation, according to the present disclosure. Methods 200 include detecting a temperature and a composition at 210, providing to a machine learning model at 220, and predicting a probability of hydrate formation at 230. Methods 200 also may include responding at 240.

Detecting the temperature and the composition at 210 may include detecting a temperature and a composition of the fluid stream. This may include detecting the temperature and the composition of any suitable phase and/or phases of the fluid stream. As an example, the detecting at 210 may include detecting a gas phase temperature and a gas phase composition of a gas phase of the fluid stream. As another example, the detecting at 210 may include detecting a liquid phase temperature and a liquid phase composition of a liquid phase of the fluid stream. As another example, the detecting at 210 may include detecting the gas phase temperature, the liquid phase temperature, the gas phase composition, and the liquid phase composition of the fluid stream.

The detecting at 210 may be performed in any suitable manner. As an example, the detecting the temperature of the fluid stream may include detecting the temperature with, via, and/or utilizing a temperature sensor, such as a thermocouple, a thermistor, a resistance temperature detector, and/or an infrared detector. As another example, the detecting the composition of the fluid stream may include detecting the composition with, via, and/or utilizing a composition sensor, such as a gas chromatograph.

The detecting at 210 also may include detecting a pressure of the fluid stream. When the detecting at 210 includes detecting the pressure of the fluid stream, the pressure may be detected in any suitable manner. As examples, the pressure may be detected with, via, and/or utilizing a pressure sensor, such as a strain gauge pressure sensor, a solid state pressure sensor, and/or a variable capacitance pressure sensor.

Providing to the machine learning model at 220 may include providing the temperature of the fluid stream and the composition of the fluid stream to the machine learning model. The machine learning model may include a machine learning model trained, or that was trained, according to and/or utilizing methods 100, which are disclosed herein. Additionally or alternatively, the machine learning model may include any suitable database, table, and/or algorithm that was generated utilizing and/or based upon the machine learning model trained according to methods 100.

Predicting the probability of hydrate formation at 230 may include predicting a statistical probability of hydrate formation within the fluid conduit and/or via the machine learning model. This may be accomplished in any suitable manner. As an example, the machine learning model may be utilized to generate a flow condition key, examples of which are disclosed herein, which may predict the statistical probability of hydrate formation within the fluid conduit at the temperature of the fluid stream and at the composition of the fluid stream. In such an example, the predicting at 230 may include comparing the temperature of the fluid stream and/or the composition of the fluid stream to the flow condition key.

As another example, and when the detecting at 210 includes detecting the gas phase temperature and the gas phase composition of the gas phase of the fluid stream, the predicting at 230 may include predicting the probability of hydrate formation based, at least in part, on the gas phase temperature and the gas phase composition. As another example, and when the detecting at 210 includes detecting the liquid phase temperature and the liquid phase composition of the liquid phase of the fluid stream, the predicting at 230 may include predicting the probability of hydrate formation based, at least in part, on the liquid phase temperature and the liquid phase composition. As yet another example, and when the detecting at 210 includes detecting the gas phase temperature, the liquid phase temperature, the gas phase composition, and the liquid phase composition, the predicting at 230 may include predicting the probability of hydrate formation based, at least in part, on the gas phase temperature, the liquid phase temperature, the gas phase composition, and the liquid phase composition. As another example, and when the detecting at 210 includes detecting the pressure of the fluid stream, the predicting at 230 may include predicting the probability of hydrate formation based, at least in part, on the pressure of the fluid stream.

Responding at 240 may include responding when, or only when, the machine learning model selectively predicts the presence of hydrate within the fluid conduit, such as during the predicting at 230. Stated differently, the responding at 240 may be responsive to the machine learning model selectively predicting the presence of hydrate within the fluid conduit.

The responding at 240 may include responding in any suitable manner. As an example, the responding at 240 may include notifying an operator of the fluid conduit of the predicted hydrate formation. As another example, the responding at 240 may include changing a flow rate of the fluid stream, such as via increasing the flow rate of the fluid stream or decreasing the flow rate of the fluid stream. As another example, the responding at 240 may include verifying at least one operating parameter of the fluid conduit, such as via verifying that the at least one operating parameter of the fluid conduit is within an expected range and/or a desired range, verifying that the fluid conduit is performing as expected, and/or verifying that an operator error has not occurred.

In some examples, methods 200 may include injecting the inhibitor, which may be configured to inhibit hydrate formation within the fluid conduit, into the fluid stream. In such examples, the responding at 240 may include changing an inhibitor injection rate of an inhibitor into the fluid stream, such as via initiating inhibitor injection and/or increasing the inhibitor injection rate. Additionally or alternatively, and in some such examples, the injecting the inhibitor may include utilizing a feedback loop to control and/or regulate the inhibitor injection rate, and the feedback loop may control and/or regulate the inhibitor injection rate based, at least in part, on the statistical probability of hydrate formation that is predicted during the predicting at 230.

When methods 200 include injecting the inhibitor, the inhibitor may be injected in any suitable manner. As an example, the injecting the inhibitor may include injecting the inhibitor with, via, and/or utilizing an inhibitor injection system, examples of which are disclosed herein with reference to inhibitor injection system 40. Examples of the inhibitor include methanol, monoethylene glycol, ethanol, diethylene glycol, propylene glycol, triethylene glycol, potassium formate, and a brine. An additional or alternative example of the inhibitor includes a mixture of a thermodynamic hydrate inhibitor and a kinetic hydrate inhibitor.

In the present disclosure, several of the illustrative, non-exclusive examples have been discussed and/or presented in the context of flow diagrams, or flow charts, in which the methods are shown and described as a series of blocks, or steps. Unless specifically set forth in the accompanying description, it is within the scope of the present disclosure that the order of the blocks may vary from the illustrated order in the flow diagram, including with two or more of the blocks (or steps) occurring in a different order and/or concurrently. It is also within the scope of the present disclosure that the blocks, or steps, may be implemented as logic, which also may be described as implementing the blocks, or steps, as logics. In some applications, the blocks, or steps, may represent expressions and/or actions to be performed by functionally equivalent circuits or other logic devices. The illustrated blocks may, but are not required to, represent executable instructions that cause a computer, processor, and/or other logic device to respond, to perform an action, to change states, to generate an output or display, and/or to make decisions.

As used herein, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B,” when used in conjunction with open-ended language such as “comprising” may refer, in one embodiment, to A only (optionally including entities other than B); in another embodiment, to B only (optionally including entities other than A); in yet another embodiment, to both A and B (optionally including other entities). These entities may refer to elements, actions, structures, steps, operations, values, and the like.

As used herein, the phrase “at least one,” in reference to a list of one or more entities should be understood to mean at least one entity selected from any one or more of the entities in the list of entities, but not necessarily including at least one of each and every entity specifically listed within the list of entities and not excluding any combinations of entities in the list of entities. This definition also allows that entities may optionally be present other than the entities specifically identified within the list of entities to which the phrase “at least one” refers, whether related or unrelated to those entities specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including entities other than B); in another embodiment, to at least one, optionally including more than one, B, with no A present (and optionally including entities other than A); in yet another embodiment, to at least one, optionally including more than one, A, and at least one, optionally including more than one, B (and optionally including other entities). In other words, the phrases “at least one,” “one or more,” and “and/or” are open-ended expressions that are both conjunctive and disjunctive in operation. For example, each of the expressions “at least one of A, B, and C,” “at least one of A, B, or C,” “one or more of A, B, and C,” “one or more of A, B, or C,” and “A, B, and/or C” may mean A alone, B alone, C alone, A and B together, A and C together, B and C together, A, B, and C together, and optionally any of the above in combination with at least one other entity.

In the event that any patents, patent applications, or other references are incorporated by reference herein and (1) define a term in a manner that is inconsistent with and/or (2) are otherwise inconsistent with, either the non-incorporated portion of the present disclosure or any of the other incorporated references, the non-incorporated portion of the present disclosure shall control, and the term or incorporated disclosure therein shall only control with respect to the reference in which the term is defined and/or the incorporated disclosure was present originally.

As used herein the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function but that the element, component, and/or other subject matter is specifically selected, created, implemented, utilized, programmed, and/or designed for the purpose of performing the function. It is also within the scope of the present disclosure that elements, components, and/or other recited subject matter that is recited as being adapted to perform a particular function may additionally or alternatively be described as being configured to perform that function, and vice versa.

As used herein, the phrase, “for example,” the phrase, “as an example,” and/or simply the term “example,” when used with reference to one or more components, features, details, structures, embodiments, and/or methods according to the present disclosure, are intended to convey that the described component, feature, detail, structure, embodiment, and/or method is an illustrative, non-exclusive example of components, features, details, structures, embodiments, and/or methods according to the present disclosure. Thus, the described component, feature, detail, structure, embodiment, and/or method is not intended to be limiting, required, or exclusive/exhaustive; and other components, features, details, structures, embodiments, and/or methods, including structurally and/or functionally similar and/or equivalent components, features, details, structures, embodiments, and/or methods, are also within the scope of the present disclosure.

As used herein, “at least substantially,” when modifying a degree or relationship, may include not only the recited “substantial” degree or relationship, but also the full extent of the recited degree or relationship. A substantial amount of a recited degree or relationship may include at least 75% of the recited degree or relationship. For example, an object that is at least substantially formed from a material includes objects for which at least 75% of the objects are formed from the material and also includes objects that are completely formed from the material. As another example, a first length that is at least substantially as long as a second length includes first lengths that are within 75% of the second length and also includes first lengths that are as long as the second length.

### INDUSTRIAL APPLICABILITY

The hydrocarbon conveyance structures and methods disclosed herein are applicable to the hydrocarbon exploration, production, and processing industries.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions, and/or properties disclosed herein. Similarly, where the claims recite “a” or “a first” element or the equivalent thereof, such claims should be understood to include incorporation of one or more such elements, neither requiring nor excluding two or more such elements.

It is believed that the following claims particularly point out certain combinations and subcombinations that are directed to one of the disclosed inventions and are novel and non-obvious. Inventions embodied in other combinations and subcombinations of features, functions, elements, and/or properties may be claimed through amendment of the present claims or presentation of new claims in this or a related application. Such amended or new claims, whether they are directed to a different invention or directed to the same invention, whether different, broader, narrower, or equal in scope to the original claims, are also regarded as included within the subject matter of the inventions of the present disclosure.

1. A method of predicting conditions indicative of hydrate formation within a fluid conduit, which contains a fluid stream that includes water and hydrocarbons, the method comprising:
for each temperature of a plurality of different temperatures of the fluid stream, determining a plurality of corresponding flow conditions of the fluid stream;
applying a plurality of test conditions to a hydrate formation model, wherein each test condition of the plurality of test conditions includes a corresponding temperature of the plurality of different temperatures and the plurality of corresponding flow conditions of the fluid stream for the corresponding temperature;
generating, from the hydrate formation model, a hydrate formation model output that predicts liquid phase composition, gaseous phase composition, and hydrate phase composition within the fluid stream for each test condition; and
training a machine learning model, utilizing the hydrate formation model output, such that, for a given temperature of the fluid stream, a given stream pressure of the fluid stream, and a given composition of the fluid stream, the machine learning model selectively predicts combinations of the liquid phase composition and the gaseous phase composition indicative of the presence of a hydrate phase within the fluid conduit.

2. The method of claim 1, wherein the plurality of different temperatures includes at least one of:
(i) a plurality of selected temperatures for the fluid stream; and
(ii) a plurality of measured temperatures measured within the fluid stream.

3. The method of claim 1, wherein the fluid stream exhibits a stream temperature variation that defines a stream temperature range, optionally wherein the stream temperature range extends between a minimum stream temperature and a maximum stream temperature.

4. The method of claim 3, wherein the stream temperature variation is at least one of:
(i) a day-to-night stream temperature variation;
(ii) a day-to-day stream temperature variation; and
(iii) a seasonal stream temperature variation.

5. The method of claim 3, wherein at least one of:
(i) the plurality of different temperatures is based, at least in part, on the stream temperature variation; and
(ii) the plurality of different temperatures is within the stream temperature variation.

6. The method of claim 3, wherein at least one of:
(i) a temperature range of the plurality of different temperatures is based, at least in part, on the stream temperature range; and
(ii) the temperature range of the plurality of different temperatures is at least substantially equal to the stream temperature range.

7. The method of claim 1, wherein the plurality of corresponding flow conditions includes at least one of:
(i) a volumetric flow rate of the fluid stream;
(ii) a flow velocity of the fluid stream; and
(iii) a flow regime of the fluid stream.

8. The method of claim 1, wherein the plurality of corresponding flow conditions includes a stream pressure of the fluid stream.

9. The method of claim 1, wherein the plurality of corresponding flow conditions includes a stream composition of the fluid stream.

10. The method of claim 9, wherein the stream composition includes a hydrocarbon fraction of the fluid stream and an aqueous fraction of the fluid stream.

11. The method of claim 10, wherein the hydrocarbon fraction of the fluid stream includes at least two of:
(i) a methane fraction of the fluid stream;
(ii) an ethane fraction of the fluid stream;
(iii) a propane fraction of the fluid stream;
(iv) a butane fraction of the fluid stream; and
(v) a pentane fraction of the fluid stream.

12. The method of claim 9, wherein the stream composition includes a hydrogen sulfide fraction of the fluid stream.

13. The method of claim 9, wherein the stream composition includes a carbon dioxide fraction of the fluid stream.

14. The method of claim 9, wherein the stream composition includes an inhibitor fraction of the fluid stream.

15. The method of claim 9, wherein the stream composition includes a nitrogen fraction of the fluid stream.

16. The method of claim 9, wherein the stream composition includes an argon fraction of the fluid stream.

17. The method of claim 1, wherein the hydrate formation model is configured to calculate the hydrate formation model output for each test condition based, at least in part, on the corresponding temperature and the plurality of corresponding flow conditions for each test condition.

18. The method of claim 1, wherein the hydrate formation model includes a flash-flow model of the fluid stream.

19. The method of claim 1, wherein the training the machine learning model includes providing the hydrate formation model output to the machine learning model as training data.

20. The method of claim 1, wherein, subsequent to the training and for a given combination of temperature and the plurality of corresponding flow conditions, the machine learning model is configured to predict a statistical probability of hydrate formation within the fluid conduit.

21. The method of claim 1, wherein, subsequent to the training and with the machine learning model, the method further includes generating a flow condition key that predicts a statistical probability of hydrate formation within the fluid conduit for a given temperature of the fluid stream and for a given composition of the fluid stream.

22. The method of claim 1, wherein, prior to the training, the method further includes superimposing a noise signal on the hydrate formation model output.

23. A method of monitoring flow of a fluid stream within a fluid conduit for hydrate formation, the method comprising:
detecting a temperature of the fluid stream and a composition of the fluid stream;
providing the temperature of the fluid stream and the composition of the fluid stream to a machine learning model trained according to the method of any of claims 1-22; and
predicting a statistical probability of hydrate formation, within the fluid conduit, via the machine learning model.

24. The method of claim 23, wherein the predicting includes comparing the temperature of the fluid stream and the composition of the fluid stream to a flow condition key that predicts a statistical probability of hydrate formation within the fluid conduit at the temperature of the fluid stream and at the composition of the fluid stream.

25.-36. (canceled)

37. A hydrocarbon conveyance structure, comprising:
a fluid tubular that defines a fluid conduit, which contains a fluid stream that includes hydrocarbons and water;
a detection device configured to detect a plurality of different temperatures of the fluid stream and a plurality of corresponding flow conditions of the fluid stream; and
a controller programmed to train a machine learning model utilizing the method of claim 1.

38.-43. (canceled)
